(19) United States
(12) Patent Application Publication
(10) Pub. No.: US 2024/0207257 A1
(43) Pub. Date: Jun. 27, 2024

(54) COMBINATION THERAPY COMPRISING A PKC INHIBITOR AND A MEK INHIBITOR

(71) Applicants: IDEAYA BIOSCIENCES, INC., South San Francisco, CA (US); PFIZER, INC., New York, NY (US)

(72) Inventors: Matthew Anthony MAURER, Princeton, NJ (US); Michael Gabriel O'QUIGLEY, Half Moon Bay, CA (US); Jaymes HOLLAND, Los Angeles, CA (US); Sarah Shwu-Kuan Jaw-Tsai, Foster City, CA (US); Julie Hambleton, South San Franciso, CA (US); MAI HOPE LE, South San Francisco, CA (US)

(21) Appl. No.: 18/555,058

(22) PCT Filed: Apr. 14, 2022
(86) PCT No.: PCT/US2022/024909

§ 371 Date: Oct. 12, 2023

Related U.S. Application Data
(60) Provisional application No. 63/175,513, filed on Apr. 15, 2021.

(51) Int. Cl. A61K 31/497; A61K 31/4184; A61P 35/00

(52) U.S. Cl. CPC A61K 31/497; A61K 31/4184; A61P 35/00

(57) ABSTRACT

Provided herein is a combination therapy and methods of using such combination therapy to treat diseases or disorders associated with PKC and MEK.

### RELATED APPLICATIONS

This application claims priority to U.S. Provisional Application No. 63/175,513, filed on Apr. 15, 2021, the entire content of which is hereby incorporated by reference in its entirety.

### BACKGROUND

Uveal melanoma is the most common primary intraocular malignant tumor in adults. Certain protein kinase inhibitors are described in International Publ. Nos. WO 02/38561 and WO 2008/106692. One protein kinase C (PKC) inhibitor, sotrastaurin, has been shown to have activity against certain PKC isototypes and has only recently been shown to selectively inhibit the growth of uveal melanoma cells harboring GNAQ mutations by targeting PKC/ERK1/2 and PKC/NF-xB pathways (see X. Wu, et al., Mol. Cancer Ther., Vol. 11, pages 1905-1914, 2012). However, there still remains an unmet need to provide next generation PKC inhibitors for treating uveal melanoma that have improved efficacy at lower dosage amounts to achieve tumor regression, improved potency, hERG activity, absorption, gastrointestinal tolerance and kinase selectivity. PCT application no. PCT/IB2015/055951 (WO 2016/020864) discloses a number of potent and selective PKC inhibitors.

Cell signaling through growth factor receptors and protein kinases is an important regulator of cell growth, proliferation and differentiation. In normal cell growth, growth factors, through receptor activation (i.e., PDGF or EGF and others), activate MAP kinase pathways. One of the most important and most well understood MAP kinase pathways involved in normal and uncontrolled cell growth is the Ras/Raf kinase pathway. Active GTP-bound Ras results in the activation and indirect phosphorylation of Raf kinase. Raf then phosphorylates MEK1 and 2 on two serine residues (S218 and S222 for MEK1 and S222 and S226 for MEK2) (Ahn, et al., Methods in Enzymology 2001, 332, 417-431). Activated MEK then phosphorylates its only known substrates, the MAP kinases, ERK1 and 2. Overall, treatment of cells with growth factors leads to the activation of ERK1 and 2 which results in proliferation and, in some cases, differentiation (Lewis et al., Adv. Cancer Res. 1998, 74, 49-139).

In proliferative diseases, genetic mutations and/or overexpression of the growth factor receptors, downstream signaling proteins, or protein kinases involved in the ERK kinase pathway lead to uncontrolled cell proliferation and, eventually, tumor formation. For example, some cancers contain mutations which result in the continuous activation of this pathway due to continuous production of growth factors. Other mutations can lead to defects in the deactivation of the activated GTP-bound Ras complex, again resulting in activation of the MAP kinase pathway.

As constitutive or overactivation of MAP kinase cascade plays a pivotal role in cell proliferation and differentiation, inhibition of this pathway is believed to be beneficial in hyperproliferative diseases. MEK is a key player in this pathway. Additionally, it is an attractive therapeutic target because the only known substrates for MEK phosphorylation are the MAP kinases, ERK1 and 2. Inhibition of MEK has been shown to have potential therapeutic benefit in several studies (U.S. Pat. No. 7,777,050). For example, small molecule MEK inhibitors have been shown to inhibit human tumor growth in nude mouse xenografts, (Sebolt-Leopold, et al., Nature-Medicine 1999, 5 (7), 810-816;), block static allodynia in animals (WO 01/05390) and inhibit growth of acute myeloid leukemia cells (Milella, et al., J. Clin. Invest., 2001, 108 (6), 851-859). PCT application no. PCT/US2003/007864 (WO 2003/077914) discloses a number of potent and selective PKC inhibitors.

Despite these developments, there still remains a need for treatment of various cancers, such as uveal melanoma.

### SUMMARY

Provided herein is a combination product comprising a PKC inhibitor and a MEK inhibitor. The combination product is useful for the treatment of a variety of cancers, including uveal melanoma. The combination product is also useful for the treatment of any number of PKC-associated and/or MEK-associated diseases.

Thus, in one aspect, provided herein is a method of treating uveal melanoma or a solid tumor harboring GNAQ or GNA11 mutations in a subject in need thereof, comprising administering to the subject Compound 1:

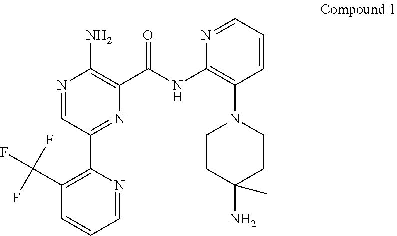 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

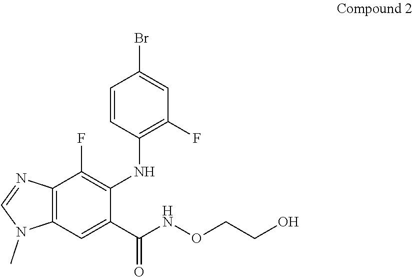 [SMILES: Cn1cnc2c(F)c(Nc3ccc(Br)cc3F)c(C(=O)NOCCO)cc21]

or a pharmaceutically acceptable salt thereof;

wherein

Compound 1, or a pharmaceutically acceptable salt thereof, is administered at a dose of about 200 mg two times per day (BID); and

Compound 2, or a pharmaceutically acceptable salt thereof, is administered at a dose of about 30 mg BID.

In another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;

wherein

Compound 1, or a pharmaceutically acceptable salt thereof, is administered at a dose of about 400 mg to about 600 mg daily; and

Compound 2, or a pharmaceutically acceptable salt thereof, is administered at a dose of about 30 mg to about 60 mg daily.

In yet another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;

according to a dosing schedule comprising at least one 7-day dosing cycle, wherein

Compound 1 or a pharmaceutically acceptable salt thereof is administered at a dose of about 200 mg BID every day of the at least one 7-day dosing cycle; and

Compound 2 or a pharmaceutically acceptable salt thereof is administered at a dose of about 30 mg BID for five consecutive days and then not administered for two consecutive days of the at least one 7-day dosing cycle.

In yet another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; according to a dosing schedule comprising at least one 7-day dosing cycle, wherein Compound 1 or a pharmaceutically acceptable salt thereof is administered at a dose of about 200 mg BID every day to about 300 BID every day of the at least one 7-day dosing cycle; and Compound 2 or a pharmaceutically acceptable salt thereof is administered at a dose of about 15 mg BID every day to about 30 mg BID every day of the at least one 7-day dosing cycle. In an embodiment, Compound 1 or a pharmaceutically acceptable salt thereof and Compound 2 or a pharmaceutically acceptable salt thereof are both continued to be administered every day after the at least one 7-day dosing cycle as disclosed herein for an additional at least two or more consecutive 7-day dosing cycles, at least four or more consecutive 7-day dosing cycles, at least eight or more consecutive 7-day dosing cycles, or at least sixteen or more consecutive 7-day dosing cycles.

In yet another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and

Compound 2, or a pharmaceutically acceptable salt thereof; according to a dosing schedule comprising at least two 7-day dosing cycles, wherein Compound 1 or a pharmaceutically acceptable salt thereof is administered at a dose of about 200 mg BID every day to about 300 BID every day of the at least two 7-day dosing cycles; and Compound 2 or a pharmaceutically acceptable salt thereof is administered at a dose of about 15 mg BID every day to about 30 mg BID every day starting on the 8th day of the at least two 7-day dosing cycles (this dosing schedule is referred to herein as a “run-in dosing schedule”). In an embodiment, Compound 1 or a pharmaceutically acceptable salt thereof and Compound 2 or a pharmaceutically acceptable salt thereof are both continued to be administered every day after the at least two 7-day dosing cycles as disclosed herein for an additional at least two or more consecutive 7-day dosing cycles, at least four or more consecutive 7-day dosing cycles, at least eight or more consecutive 7-day dosing cycles, or at least sixteen or more consecutive 7-day dosing cycles.

In yet another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; according to a dosing schedule comprising at least one 7-day dosing cycle, wherein Compound 1 or a pharmaceutically acceptable salt thereof is administered at a dose of about 200 mg BID every day to about 300 BID every day of the at least one 7-day dosing cycle; and Compound 2 or a pharmaceutically acceptable salt thereof is administered at a dose of about 15 mg BID every day to about 30 mg BID for five consecutive days and then not administered for two consecutive days of the at least one 7-day dosing cycle (this dosing schedule is referred to herein as an “intermittent dosing schedule”). In an embodiment, Compound 1 or a pharmaceutically acceptable salt thereof is continued to be administered every day and Compound 2 or a pharmaceutically acceptable salt thereof is continued to be administered for five consecutive days and then not administered for two consecutive days of the at least one 7-day dosing cycle as disclosed herein for an additional two or more consecutive 7-day dosing cycles, four or more consecutive 7-day dosing cycles, eight or more consecutive 7-day dosing cycles, or sixteen or more consecutive 7-day dosing cycles.

In an embodiment, the uveal melanoma is metastatic uveal melanoma. The uveal melanoma can be a solid tumor harboring GNAQ or GNA11 mutations. In another embodiment, the solid tumor harboring GNAQ or GNA11 mutations to be treated is cutaneous melanoma. In another embodiment, the solid tumor harboring GNAQ or GNA11 mutations to be treated is non-small cell lung cancer (NSCLC).

In another embodiment, Compound 1, or a pharmaceutically acceptable salt thereof, is administered at a dose of 200 mg BID. In another embodiment, Compound 2, or a pharmaceutically acceptable salt thereof, is administered at a dose of 30 mg BID. In yet another embodiment, according to a dosing schedule comprising at least one 7-day dosing cycle, Compound 2 or a pharmaceutically acceptable salt thereof is administered at a dose of about 30 mg BID for five consecutive days and then not administered for two consecutive days of the at least one 7-day dosing cycle. In still another embodiment, the administration of Compound 1, or a pharmaceutically acceptable salt thereof, in combination with the administration of Compound 2, or a pharmaceutically acceptable salt thereof, is continued uninterrupted during a dosing schedule comprising at least one 7-day dosing cycle.

With respect to any of the aforementioned embodiments, Compound 1 or a pharmaceutically acceptable salt thereof is administered at a dose of about 200 mg BID and Compound 2 or a pharmaceutically acceptable salt thereof is administered at a dose of about 30 mg BID.

With respect to any of the aforementioned embodiments, Compound 1 or a pharmaceutically acceptable salt thereof is administered at a dose of about 300 mg BID and Compound 2 or a pharmaceutically acceptable salt thereof is administered at a dose of about 15 mg BID.

With respect to any of the aforementioned embodiments, Compound 1 or a pharmaceutically acceptable salt thereof is administered at a dose of about 300 mg BID and Compound 2 or a pharmaceutically acceptable salt thereof is administered at a dose of about 30 mg BID.

With respect to any of the aforementioned embodiments, the method of treating uveal melanoma or a solid tumor harboring GNAQ or GNA11 mutations in a subject in need thereof can further comprise: (i) following administration of Compound 1 and Compound 2 to the patient in need thereof according to the dosing regimen of any of the aforementioned embodiments over a treatment period comprising at least 4 consecutive 7-day dosing cycles, then (ii) increasing the dose of Compound 1 to 300 mg BID or 600 mg total daily as applicable, and/or (iii) lowering the dose of Compound 2 to 15 mg BID or 30 mg total daily as applicable.

With respect to any of the aforementioned embodiments, the method of treating uveal melanoma or a solid tumor harboring GNAQ or GNA11 mutations in a subject in need thereof can further comprise: continuing to administer Compound 1, or a pharmaceutically acceptable salt thereof, and Compound 2, or a pharmaceutically acceptable salt thereof, uninterrupted during a dosing schedule comprising at least two or more consecutive 7-day dosing cycles, or preferably at least four or more consecutive 7-day dosing cycles. In particularly preferred embodiments, Compound 1, or a pharmaceutically acceptable salt thereof, and Compound 2, or a pharmaceutically acceptable salt thereof, are continued to be co-administered uninterrupted during a dosing schedule comprising at least eight or more, twelve or more, twenty-four or more, forty-eight or more or ninety-six or more consecutive 7-day dosing cycles.

In another aspect, provided herein is a combination product comprising
(i) about 200 mg Compound 1, or a pharmaceutically acceptable salt thereof; and
(ii) about 30 mg of Compound 2, or a pharmaceutically acceptable salt thereof.

In yet another embodiment, provided herein is a combination product comprising
(i) about 200 mg to about 600 mg Compound 1, or a pharmaceutically acceptable salt thereof; and
(ii) about 15 mg to about 60 mg of Compound 2, or a pharmaceutically acceptable salt thereof.

In another aspect, provided herein is a combination product comprising
(i) about 300 mg Compound 1, or a pharmaceutically acceptable salt thereof; and
(ii) about 30 mg of Compound 2, or a pharmaceutically acceptable salt thereof.

In another aspect, provided herein is a combination product comprising
(i) about 300 mg Compound 1, or a pharmaceutically acceptable salt thereof; and
(ii) about 15 mg of Compound 2, or a pharmaceutically acceptable salt thereof.

In an embodiment, the combination product comprises Compound 1 as a free base. In another embodiment, the combination product comprises Compound 2 as a free base.

### BRIEF DESCRIPTION OF THE DRAWINGS

FIG. 1A and FIG. 1B. CT scans of Patient 9. Patient 9 with advanced MUM with multiple metastatic liver lesions dosed with Compound 1 at 200 mg bid and Compound 2 at 30 mg bid on continuous schedule. First imaging assessment shows a 20.5% reduction in some of target lesions.

### DETAILED DESCRIPTION

Provided herein is a combination therapy comprising Compound 1, or a pharmaceutically acceptable salt thereof, and Compound 2, or a pharmaceutically acceptable salt thereof. The combination therapy is useful for the treatment of a variety of cancers, including uveal melanoma. The combination therapy can also be useful for treatment of a solid tumor harboring GNAQ or GNA11 mutations. In another aspect, the combination therapy is useful for the treatment of any number of PKC-associated and/or MEK-associated diseases.

Administering a combination of Compound 1 and a Compound 2 can provide beneficial effects for treating cancer, e.g., uveal melanoma, in a subject. Such an approach—combination or co-administration of the two types of agents—may offer an uninterrupted treatment to an subject in need over a clinically relevant treatment period.

### Definitions

Listed below are definitions of various terms used herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

As used in the specification and in the claims, the term “comprising” may include the embodiments “consisting of” and “consisting essentially of.” The terms “comprise(s),” “include(s),” “having,” “has,” “may,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically acceptable carriers, and excludes other compounds.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt. % to about 5 wt. %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, of the numerical value(s) being modified. In addition, the phrase “about ‘x’ to ‘y’” includes “about ‘x’ to about ‘y’”.

The terms “combination,” “therapeutic combination,” “pharmaceutical combination,” or “combination product” as used herein refer to either a fixed combination in one dosage unit form, or non-fixed combination in separate dosage forms, or a kit of parts for the combined administration where two or more therapeutic agents may be administered independently, at the same time or separately within time intervals.

The term “combination therapy” refers to the administration of two or more therapeutic agents to treat a therapeutic condition or disorder described in the present disclosure. Such administration encompasses co-administration of these therapeutic agents in a substantially simultaneous manner, such as in a single formulation having a fixed ratio of active ingredients or in separate formulations (e.g., capsules and/or intravenous formulations) for each active ingredient. In addition, such administration also encompasses use of each type of therapeutic agent in a sequential or separate manner, either at approximately the same time or at different times. Regardless of whether the active ingredients are administered as a single formulation or in separate formulations, the drugs are administered to the same patient as part of the same course of therapy. In any case, the treatment regimen will provide beneficial effects in treating the conditions or disorders described herein.

As used herein, the term “treating” or “treatment” refers to one or more of (1) preventing the disease; for example, preventing a disease, condition or disorder in an individual who may be predisposed to the disease, condition or disorder but does not yet experience or display the pathology or symptomatology of the disease; (2) inhibiting the disease; for example, inhibiting a disease, condition or disorder in an individual who is experiencing or displaying the pathology or symptomatology of the disease, condition or disorder (i.e., arresting further development of the pathology and/or symptomatology); and (3) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease. In some embodiments, the term “treating” or “treatment” refers to inhibiting or ameliorating the disease.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein a parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts described herein include the conventional non-toxic salts of the parent compound formed, for example, from non-toxic inorganic or organic acids. The pharmaceutically acceptable salts discussed herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are used. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound with a pharmaceutically acceptable carrier. The pharmaceutical composition facilitates administration of the composition to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound disclosed herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of a compound disclosed herein, and are physiologically acceptable to the patient. Supplementary active compounds may also be incorporated into the compositions. The “pharmaceutically acceptable carrier” may further include a pharmaceutically acceptable salt of the compound(s) disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

The term “single formulation” as used herein refers to a single carrier or vehicle formulated to deliver effective amounts of both therapeutic agents to a patient. The single vehicle is designed to deliver an effective amount of each of the agents, along with any pharmaceutically acceptable carriers or excipients. In some embodiments, the vehicle is a tablet, capsule, pill, or a patch. In other embodiments, the vehicle is a solution or a suspension.

The term “unit dose” is used herein to mean simultaneous administration of both agents together, in one dosage form, to the patient being treated. In some embodiments, the unit dose is a single formulation. The term “a unit dose,” as used herein can also refer to the simultaneous administration of both agents separately, in two dosage forms, to the patient being treated. In certain embodiments, the unit dose includes one or more vehicles such that each vehicle includes an effective amount of at least one of the agents along with pharmaceutically acceptable carriers and excipients. In some embodiments, the unit dose is one or more tablets, capsules, pills, or patches administered to the patient at the same time.

The combination of agents described herein may display a synergistic effect. See, for example, Frey, C. R., et al., Analysis of drug combinations with the PKC inhibitor IDE196 support dual MEK and PKC inhibition as a rational combination in metastatic uveal melanoma, AACR Meeting 2019, the entire content of which is hereby incorporated by reference. The term “synergistic effect” as used herein, refers to action of two agents such as, for example, Compound 1 and Compound 2, producing an effect, for example, slowing the symptomatic progression of cancer or symptoms thereof, which is greater than the simple addition of the effects of each drug administered by themselves. A synergistic effect can be calculated, for example, using suitable methods such as the Sigmoid-Emax equation (Holford, N. H. G. and Scheiner, L. B., Clin. Pharmacokinet. 6: 429-453 (1981)), the equation of Loewe additivity (Loewe, S. and Muischnek, H., Arch. Exp. Pathol Pharmacol. 114: 313-326 (1926)) and the median-effect equation (Chou, T. C. and Talalay, P., Adv. Enzyme Regul. 22: 27-55 (1984)). Each equation referred to above can be applied to experimental data to generate a corresponding graph to aid in assessing the effects of the drug combination. The corresponding graphs associated with the equations referred to above are the concentration-effect curve, isobologram curve and combination index curve, respectively.

As used herein, the term “synergy” refers to the effect achieved when the active ingredients, i.e., Compound 1 and Compound 2, used together is greater than the sum of the effects that results from using the compounds separately.

In an embodiment, provided herein is a combination therapy comprising an effective amount of Compound 1 and Compound 2. An “effective amount” of a combination of agents (i.e., Compound 1 and Compound 2) is an amount sufficient to provide an observable improvement over the baseline clinically observable signs and symptoms of the disorders treated with the combination.

An “oral dosage form” includes a unit dosage form prescribed or intended for oral administration.

### Combination Product

Provided herein is a combination product comprising Compound 1, or a pharmaceutically acceptable salt thereof, and Compound 2, or a pharmaceutically acceptable salt thereof. The combination product is useful for the treatment of a variety of cancers, including uveal melanoma, for example uveal melanoma harboring GNAQ or GNA11 mutations; or metastatic uveal melanoma. The combination product can also be useful for treatment of a solid tumor harboring GNAQ or GNA11 mutations. In another aspect, the combination product is useful for the treatment of any number of PKC-associated and/or MEK-associated diseases.

The compound 3-amino-N-(3-(4-amino-4-methylpiperidin-1-yl)pyridin-2-yl)-6-(3-(trifluoromethyl)pyridin-2-yl)pyrazine-2-carboxamide (also known as 3-amino-N-[3-(4-amino-4-methyl-1-piperidinyl)-2-pyridinyl]-6-[3-(trifluoromethyl)-2-pyridinyl]-2-pyrazinecarboxamide) having the structure:

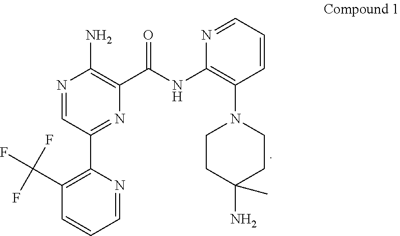 [SMILES: CC1(N)CCN(c2cccnc2NC(=O)c2nc(-c3ncccc3C(F)(F)F)cnc2N)CC1]

Compound 1 is a potent and selective inhibitor of protein kinase C. Compound 1, its synthesis, and biological activity against PKC can be found in PCT/IB2015/055951 (WO2016020864), which is incorporated by reference in its entirety. Compound 1 has been assigned the International Nonproprietary Name (INN) darovasertib.

The compound 5-[(4-bromo-2-fluorophenyl)amino]-4-fluoro-N-(2-hydroxyethoxy)-1-methyl-1H-benzimidazole-6-carboxamide (also known as 6-(4-bromo-2-fluoro-phenylamino)-7-fluoro-3-methyl-3H-benzoimidazole-5-carboxylic acid (2-hydroxy-ethoxy)-amide) having the structure:

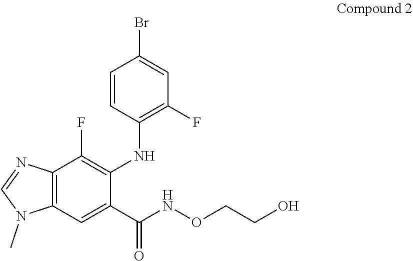 [SMILES: Cn1cnc2c(F)c(Nc3ccc(Br)cc3F)c(C(=O)NOCCO)cc21]

has been assigned the International Nonproprietary Name (INN) binimetinib. Compound 2 is a potent and selective inhibitor of MEK. Compound 2, its synthesis, and biological activity against MEK can be found in PCT/US2003/007864 (WO2003077914), which is incorporated by reference in its entirety.

Thus, in an aspect, provided herein is a combination product comprising
(i) about 200 mg Compound 1:

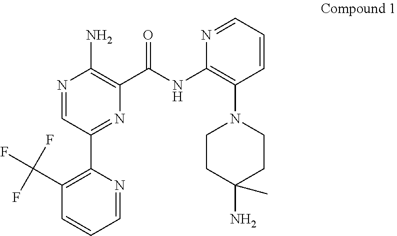 [SMILES: CC1(N)CCN(c2cccnc2NC(=O)c2nc(-c3ncccc3C(F)(F)F)cnc2N)CC1]

or a pharmaceutically acceptable salt thereof; and
(ii) about 30 mg of Compound 2:

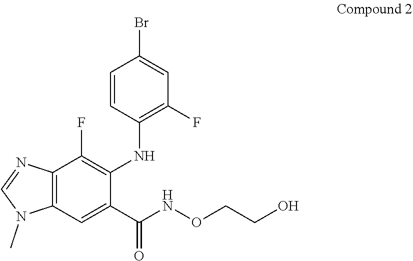 [SMILES: Cn1cnc2c(F)c(Nc3ccc(Br)cc3F)c(C(=O)NOCCO)cc21]

or a pharmaceutically acceptable salt thereof. In another aspect, provided herein is a combination product comprising (i) about 200 mg to about 600 mg Compound 1:

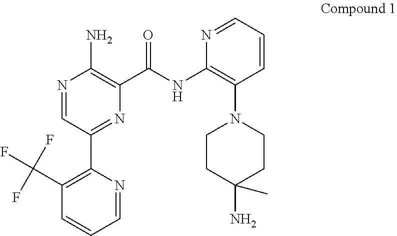 [SMILES: CC1(N)CCN(c2cccnc2NC(=O)c2nc(-c3ncccc3C(F)(F)F)cnc2N)CC1]

or a pharmaceutically acceptable salt thereof; and
(ii) about 15 mg to about 60 mg of Compound 2:

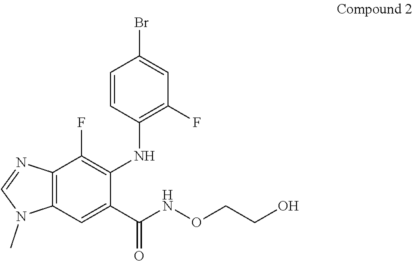 [SMILES: Cn1cnc2c(F)c(Nc3ccc(Br)cc3F)c(C(=O)NOCCO)cc21]

or a pharmaceutically acceptable salt thereof.

In an embodiment, the combination product provided above comprises about 200 mg of Compound 1, or a pharmaceutically acceptable salt thereof. In another embodiment, the combination product comprises about 30 mg of Compound 2, or a pharmaceutically acceptable salt thereof.

In an embodiment, the combination product provided above comprises about 300 mg of Compound 1, or a pharmaceutically acceptable salt thereof. In another embodiment, the combination product comprises about 15 mg of Compound 2, or a pharmaceutically acceptable salt thereof.

In an embodiment, the combination product provided above comprises about 300 mg of Compound 1, or a pharmaceutically acceptable salt thereof. In another embodiment, the combination product comprises about 30 mg of Compound 2, or a pharmaceutically acceptable salt thereof.

In another embodiment, the combination product comprises Compound 1 as a free base. In yet another embodiment, the combination product comprises Compound 2 as a free base.

In an embodiment, Compound 1, or a pharmaceutically acceptable salt thereof, is present in the combination product at a dose selected from the group consisting of 200 mg, 250 mg, 300 mg, 350 mg, 400 mg, 450 mg, 500 mg, 550 mg, and 600 mg. In another embodiment, Compound 1, or a pharmaceutically acceptable salt thereof, is present in the combination product at a dose of 200 mg. In yet another embodiment, Compound 1, or a pharmaceutically acceptable salt thereof, is present in the combination product at a dose of 300 mg. In still another embodiment, Compound 1, or a pharmaceutically acceptable salt thereof, is orally administered as a tablet. In another embodiment, Compound 1, or a pharmaceutically acceptable salt thereof, is administered twice daily (BID).

In another embodiment, Compound 1, or a pharmaceutically acceptable salt thereof, is present in the combination product at a dose of 200 mg once daily (QD). In yet another embodiment, Compound 1, or a pharmaceutically acceptable salt thereof, is present in the combination product at a dose of 300 mg once daily (QD).

In an embodiment, Compound 2, or a pharmaceutically acceptable salt thereof, is present in the combination product at a dose selected from the group consisting of 5 mg, 10 mg, 15 mg, 20 mg, 25 mg, 30 mg, 35 mg, 40 mg, 45 mg, 50 mg, 55 mg, and 60 mg. In another embodiment, Compound 2, or a pharmaceutically acceptable salt thereof, is present in the combination product at a dose of 15 mg. In yet another embodiment, Compound 2, or a pharmaceutically acceptable salt thereof, is present in the combination product at a dose of 30 mg. In still another embodiment, Compound 2, or a pharmaceutically acceptable salt thereof, is orally administered as a tablet. In another embodiment, Compound 2, or a pharmaceutically acceptable salt thereof, is administered twice daily (BID).

In another embodiment, Compound 2, or a pharmaceutically acceptable salt thereof, is present in the combination product at a dose of 15 mg once daily (QD). In yet another embodiment, Compound 2, or a pharmaceutically acceptable salt thereof, is present in the combination product at a dose of 30 mg once daily (QD).

In an embodiment, Compound 1, or a pharmaceutically acceptable salt thereof, is present in the combination product at a dose of 200 mg BID, and Compound 2, or a pharmaceutically acceptable salt thereof, is present in the combination product at a dose of 15 mg BID. In another embodiment, Compound 1, or a pharmaceutically acceptable salt thereof, is present in the combination product at a dose of 300 mg BID, and Compound 2, or a pharmaceutically acceptable salt thereof, is present in the combination product at a dose of 15 mg BID. In yet another embodiment, Compound 1, or a pharmaceutically acceptable salt thereof, is present in the combination product at a dose of 200 mg BID, and Compound 2, or a pharmaceutically acceptable salt thereof, is present in the combination product at a dose of 30 mg BID. In yet another embodiment, Compound 1, or a pharmaceutically acceptable salt thereof, is present in the combination product at a dose of 300 mg BID, and Compound 2, or a pharmaceutically acceptable salt thereof, is present in the combination product at a dose of 30 mg BID.

In one embodiment, any of the above described combination products is a non-fixed combination product.

In one embodiment of any of the above described combination products, Compound 1 is 3-amino-N-(3-(4-amino-4-methylpiperidin-1-yl)pyridin-2-yl)-6-(3-(trifluoromethyl)pyridin-2-yl)pyrazine-2-carboxamide.

In one embodiment of any of the above described combination products, Compound 2 is 5-[(4-bromo-2-fluorophenyl)amino]-4-fluoro-N-(2-hydroxyethoxy)-1-methyl-1H-benzimidazole-6-carboxamide.

The administration of a pharmaceutical combination provided herein may result in a beneficial effect, e.g. a synergistic therapeutic effect, e.g., with regard to alleviating, delaying progression of or inhibiting the symptoms, and may also result in further surprising beneficial effects, e.g., fewer side-effects, an improved quality of life or a decreased morbidity, compared with a monotherapy applying only one of the pharmaceutically active ingredients used in the combination of the invention.

### Methods of Treatment

In an aspect, provided herein is a method of treating uveal melanoma or a solid tumor harboring GNAQ or GNA11 mutations in a subject in need thereof, comprising administering to the subject Compound 1:

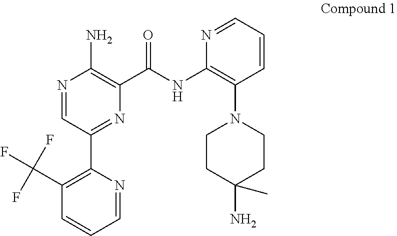 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

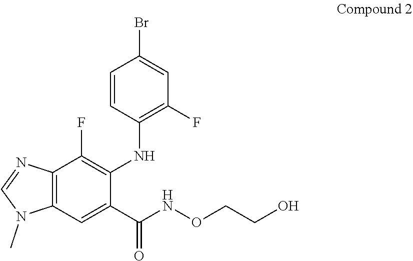 [SMILES: Cn1cnc2c(F)c(Nc3ccc(Br)cc3F)c(C(=O)NOCCO)cc21]

or a pharmaceutically acceptable salt thereof;

wherein

Compound 1 or a pharmaceutically acceptable salt thereof is administered at a dose of about 200 mg two times per day (BID); and

Compound 2 or a pharmaceutically acceptable salt thereof is administered at a dose of about 30 mg BID.

In another aspect, provided herein is a method of treating uveal melanoma or a solid tumor harboring GNAQ or GNA11 mutations in a subject in need thereof, comprising administering to the subject Compound 1or a pharmaceutically acceptable salt thereof, and Compound 2 or a pharmaceutically acceptable salt thereof; wherein Compound 1, or a pharmaceutically acceptable salt thereof, is administered at a dose of about 300 mg two times per day (BID); and Compound 2, or a pharmaceutically acceptable salt thereof, is administered at a dose of about 30 mg two times per day (BID).

In another aspect, provided herein is a method of treating uveal melanoma or a solid tumor harboring GNAQ or GNA11 mutations in a subject in need thereof, comprising administering to the subject Compound 1or a pharmaceutically acceptable salt thereof, and Compound 2 or a pharmaceutically acceptable salt thereof; wherein Compound 1, or a pharmaceutically acceptable salt thereof, is administered at a dose of about 300 mg two times per day (BID); and Compound 2, or a pharmaceutically acceptable salt thereof, is administered at a dose of about 15 mg two times per day (BID).

In another aspect, provided herein is a method of treating uveal melanoma or a solid tumor harboring GNAQ or GNA11 mutations in a subject in need thereof, comprising administering to the subject Compound 1:

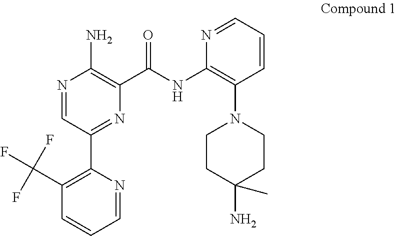 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

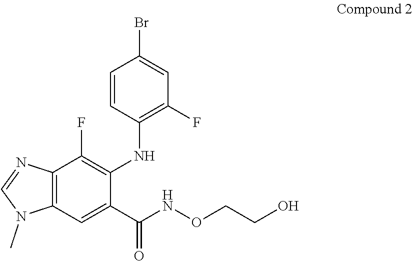 [SMILES: Cn1cnc2c(F)c(Nc3ccc(Br)cc3F)c(C(=O)NOCCO)cc21]

or a pharmaceutically acceptable salt thereof;

wherein

Compound 1, or a pharmaceutically acceptable salt thereof, is administered at a dose of about 400 mg to about 600 mg daily; and

Compound 2, or a pharmaceutically acceptable salt thereof, is administered at a dose of about 30 mg to about 60 mg daily.

In yet another aspect, provided herein is a method of treating uveal melanoma or a solid tumor harboring GNAQ or GNA11 mutations in a subject in need thereof, comprising administering to the subject Compound 1:

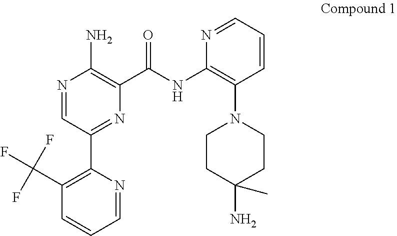 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

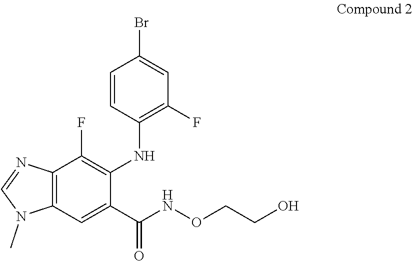 [SMILES: Cn1cnc2c(F)c(Nc3ccc(Br)cc3F)c(C(=O)NOCCO)cc21]

or a pharmaceutically acceptable salt thereof;

according to a dosing schedule comprising at least one 7-day dosing cycle, wherein

Compound 1 or a pharmaceutically acceptable salt thereof is administered at a dose of about 200 mg BID every day of the at least one 7-day dosing cycle; and

Compound 2 or a pharmaceutically acceptable salt thereof is administered at a dose of about 30 mg BID for five consecutive days and then not administered for two consecutive days of the at least one 7-day dosing cycle.

In still another aspect, provided herein is a method of treating uveal melanoma or a solid tumor harboring GNAQ or GNA11 mutations in a subject in need thereof, comprising administering to the subject Compound 1:

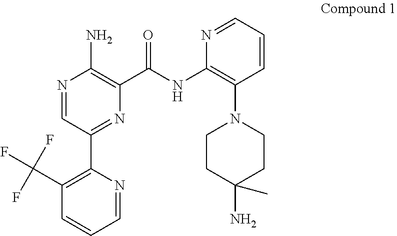 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

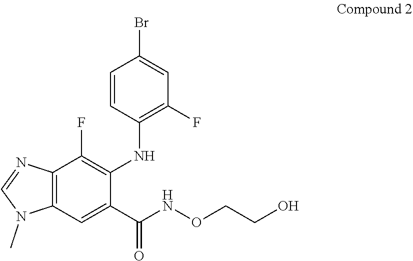 [SMILES: Cn1cnc2c(F)c(Nc3ccc(Br)cc3F)c(C(=O)NOCCO)cc21]

or a pharmaceutically acceptable salt thereof;

wherein

Compound 1, or a pharmaceutically acceptable salt thereof, is administered at a dose of about 200 mg; and

Compound 2, or a pharmaceutically acceptable salt thereof, is administered at a dose of about 30 mg.

In yet another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; according to a dosing schedule comprising at least one 7-day dosing cycle, wherein Compound 1 or a pharmaceutically acceptable salt thereof is administered at a dose of about 200 mg BID every day to about 300 BID every day of the at least one 7-day dosing cycle; and Compound 2 or a pharmaceutically acceptable salt thereof is administered at a dose of about 15 mg BID every day to about 30 mg BID every day of the at least one 7-day dosing cycle.

In yet another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; according to a dosing schedule comprising at least two 7-day dosing cycles, wherein Compound 1 or a pharmaceutically acceptable salt thereof is administered at a dose of about 200 mg BID every day to about 300 BID every day of the at least two 7-day dosing cycles; and Compound 2 or a pharmaceutically acceptable salt thereof is administered at a dose of about 15 mg BID every day to about 30 mg BID every day starting on the 8^{th} day of the at least two 7-day dosing cycles. In an embodiment, Compound 1 or a pharmaceutically acceptable salt thereof and Compound 2 or a pharmaceutically acceptable salt thereof are both administered every day after the at least two 7-day dosing cycles as disclosed herein for an additional at least two or more consecutive 7-day dosing cycles, at least four or more consecutive 7-day dosing cycles, or at least eight or more consecutive 7-day dosing cycles.

In yet another aspect, provided herein is a method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof; according to a dosing schedule comprising at least one 7-day dosing cycle, wherein Compound 1 or a pharmaceutically acceptable salt thereof is administered at a dose of about 200 mg BID every day to about 300 BID every day of the at least one 7-day dosing cycle; and Compound 2 or a pharmaceutically acceptable salt thereof is administered at a dose of about 15 mg BID every day to about 30 mg BID for five consecutive days and then not administered for two consecutive days of the at least one 7-day dosing cycle.

In an embodiment, the uveal melanoma is metastatic uveal melanoma. In another embodiment, the uveal melanoma is a solid tumor harboring GNAQ or GNA11 mutations.

In yet another embodiment, the solid tumor harboring GNAQ or GNA11 mutations is cutaneous melanoma.

In still another embodiment, Compound 1, or a pharmaceutically acceptable salt thereof, is administered at a dose of about 200 mg BID. In an embodiment, Compound 1, or a pharmaceutically acceptable salt thereof, is administered at a dose of about 300 mg BID. In another embodiment, Compound 1, or a pharmaceutically acceptable salt thereof, is administered at a dose of about 200 mg once daily (QD). In yet another embodiment, Compound 1, or a pharmaceutically acceptable salt thereof, is administered at a dose of about 300 mg once daily (QD).

In another embodiment, Compound 2, or a pharmaceutically acceptable salt thereof, is administered at a dose of about 30 mg BID. In an embodiment, Compound 2, or a pharmaceutically acceptable salt thereof, is administered at a dose of about 15 mg BID. In another embodiment, Compound 2, or a pharmaceutically acceptable salt thereof, is administered at a dose of about 15 mg once daily (QD). In yet another embodiment, Compound 2, or a pharmaceutically acceptable salt thereof, is administered at a dose of about 30 mg once daily (QD).

In an embodiment, according to a dosing schedule comprising at least one 7-day dosing cycle, Compound 2 or a pharmaceutically acceptable salt thereof is administered at a dose of about 30 mg BID for five consecutive days and then not administered for two consecutive days of the at least one 7-day dosing cycle.

In an embodiment, according to a dosing schedule comprising at least two 7-day dosing cycles, Compound 2 or a pharmaceutically acceptable salt thereof is administered at a dose of about 15 mg BID every day to about 30 mg BID every day starting on the 8^{th} day of the at least two 7-day dosing cycles. In yet another embodiment, Compound 2 or a pharmaceutically acceptable salt thereof is administered every day after the at least two 7-day dosing cycles as disclosed herein for an additional at least two or more consecutive 7-day dosing cycles, at least four or more consecutive 7-day dosing cycles, or at least eight or more consecutive 7-day dosing cycles.

In another embodiment, the administration of Compound 1, or a pharmaceutically acceptable salt thereof, is uninterrupted during a dosing schedule comprising at least one 7-day dosing cycle.

In another embodiment, the administration of Compound 2, or a pharmaceutically acceptable salt thereof, is uninterrupted during a dosing schedule comprising at least one 7-day dosing cycle.

In yet another embodiment, Compound 1, or a pharmaceutically acceptable salt thereof, and Compound 2, or a pharmaceutically acceptable salt thereof, are administered in a single formulation. In still another embodiment, Compound 1, or a pharmaceutically acceptable salt thereof, and Compound 2, or a pharmaceutically acceptable salt thereof, are administered in a single formulation further comprising one or more pharmaceutically acceptable carriers.

In an embodiment, Compound 1, or a pharmaceutically acceptable salt thereof, and Compound 2, or a pharmaceutically acceptable salt thereof, are administered separately.

In another embodiment, the treatment comprises administering the Compound 1, or a pharmaceutically acceptable salt thereof, and Compound 2, or a pharmaceutically acceptable salt thereof, at substantially the same time. In yet another embodiment, the treatment comprises administering Compound 1, or a pharmaceutically acceptable salt thereof, and Compound 2, or a pharmaceutically acceptable salt thereof, at different times.

In still another embodiment, Compound 1, or a pharmaceutically acceptable salt thereof, is administered to the subject, followed by administration of Compound 2, or a pharmaceutically acceptable salt thereof. In an embodiment, Compound 2, or a pharmaceutically acceptable salt thereof, is administered to the subject, followed by administration of Compound 1, or a pharmaceutically acceptable salt thereof.

In an embodiment, administering Compound 1, or a pharmaceutically acceptable salt thereof, and Compound 2, or a pharmaceutically acceptable salt thereof, reduces the size of one or more lesions of the uveal melanoma or the solid tumor harboring GNAQ or GNA11 mutations in said subject.

In another embodiment, administering Compound 1, or a pharmaceutically acceptable salt thereof, and Compound 2, or a pharmaceutically acceptable salt thereof, decreases the growth rate of one of more lesions of the uveal melanoma or the solid tumor harboring GNAQ or GNA11 mutations in said subject.

In yet another embodiment, the method comprises administering to the subject in need thereof Compound 1:

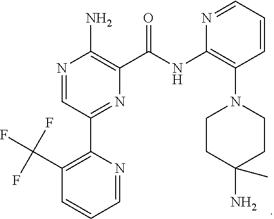 [SMILES: CC1(N)CCN(c2cccnc2NC(=O)c2nc(-c3ncccc3C(F)(F)F)cnc2N)CC1]

In still another embodiment, the method comprises administering to the subject in need thereof Compound 2:

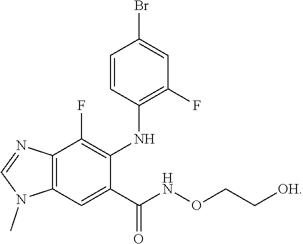 [SMILES: Cn1cnc2c(F)c(Nc3ccc(Br)cc3F)c(C(=O)NOCCO)cc21]

In an embodiment, Compound 1, or a pharmaceutically acceptable salt thereof, and Compound 2, or a pharmaceutically acceptable salt thereof, are administered orally.

In another embodiment, the method of treating uveal melanoma or a solid tumor harboring GNAQ or GNA11 mutations in a subject in need thereof can further comprise: (i) following administration of Compound 1, or a pharmaceutically acceptable salt thereof, and Compound 2, or a pharmaceutically acceptable salt thereof, to the patient in need thereof according to the dosing regimen of any of the aforementioned embodiments over a treatment period comprising at least 4 consecutive 7-day dosing cycles, then (ii) increasing the dose of Compound 1, or a pharmaceutically acceptable salt thereof, to 300 mg BID or 600 mg total daily as applicable, and/or (iii) lowering the dose of Compound 2, or a pharmaceutically acceptable salt thereof, to 15 mg BID or 30 mg total daily as applicable.

In yet another embodiment, the method of treating uveal melanoma or a solid tumor harboring GNAQ or GNA11 mutations in a subject in need thereof can further comprise: continuing to administer Compound 1, or a pharmaceutically acceptable salt thereof, and Compound 2, or a pharmaceutically acceptable salt thereof, uninterrupted during a dosing schedule comprising at least two or more consecutive 7-day dosing cycles, or preferably at least four or more consecutive 7-day dosing cycles. In particularly preferred embodiments, Compound 1, or a pharmaceutically acceptable salt thereof, and Compound 2, or a pharmaceutically acceptable salt thereof, are continued to be co-administered uninterrupted during a dosing schedule comprising at least eight or more, twelve or more, twenty-four or more, forty-eight or more or ninety-six or more consecutive 7-day dosing cycles. In an embodiment, Compound 1, or a pharmaceutically acceptable salt thereof, is continued to be co-administered uninterrupted during a dosing schedule comprising at least eight or more, twelve or more, twenty-four or more, forty-eight or more or ninety-six or more consecutive 7-day dosing cycles. In another embodiment, Compound 2, or a pharmaceutically acceptable salt thereof, is continued to be co-administered uninterrupted during a dosing schedule comprising at least eight or more, twelve or more, twenty-four or more, forty-eight or more or ninety-six or more consecutive 7-day dosing cycles.

The GNAQ or GNA11 tumor to be treated can include one or more of a number of mutations, including a substitution mutation, an insertion mutation, and/or a deletion in GNAQ or GNA11 mutation. In some aspects, the GNAQ or GNA11 mutation is a gain of function mutation. In some aspects, the GNAQ or GNA11 mutation activates the PKC signaling pathway. In various aspects, the GNAQ or GNA11 mutation can be the substitution of glutamine in codon 209 (Q209) and/or a substitution of arginine in codon 183 (R183). The GNAQ or GNA11 mutation can be a substitution other than glutamine in codon 209 (Q209), other than a substitution of arginine in codon 183 (R183), or other than both. In some aspects, the GNAQ mutation is one of Q209P, Q209L, Q209H, Q209K, or Q209Y, or the GNA11 mutation is one of Q209P, Q209L, Q209K or Q209H. In further aspects, the GNAQ mutation can be R183Q, or the GNA11 mutation can be R183C or R183H. In yet further examples, the GNAQ or GNA11 mutation is at one or more of R256, L279, R166, A168, R210, R213, R166, A231, A342, D333, G171, R147, R73, T47, E191, E221, R149, T175, T379, T85, A86, E163, D195, E319, E191, E280, E49, P293, R300, R338, R60, D155, D205, D321, I226, R37, or V240. In further examples, the GNAQ or GNA11 tumor can comprise one or more of a Q209P, Q209L, Q209H, Q209K, Q209Y, or R183Q mutation in GNAQ, or the GNAQ or GNA11 tumor can comprise one or more of a Q209P, Q209L, Q209H, or Q209K mutation in GNA11. Additional examples of mutations in GNAQ or GNA11 are described in WO 2020/146355, which is incorporated by reference herewith in its entirety.

In another aspect, provided herein is a method of treating cancer in a subject in need thereof, comprising administering to the subject Compound 1:

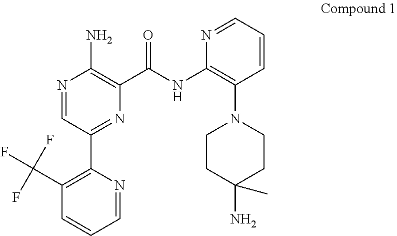 [SMILES: CC1(N)CCN(c2cccnc2NC(=O)c2nc(-c3ncccc3C(F)(F)F)cnc2N)CC1]

or a pharmaceutically acceptable salt thereof,

### and Compound 2:

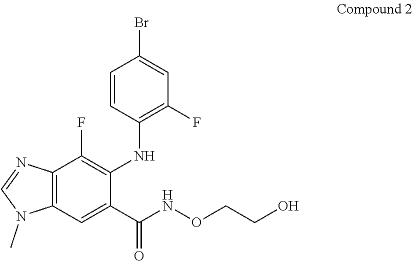 [SMILES: Cn1cnc2c(F)c(Nc3ccc(Br)cc3F)c(C(=O)NOCCO)cc21]

or a pharmaceutically acceptable salt thereof;

wherein

Compound 1 or a pharmaceutically acceptable salt thereof is administered at a dose of about 400 mg to about 600 mg daily; and

Compound 2 or a pharmaceutically acceptable salt thereof is administered at a dose of about 30 mg to about 60 mg daily.

In an embodiment, Compound 1 or a pharmaceutically acceptable salt thereof is administered at a dose of about 200 mg BID, and Compound 2 or a pharmaceutically acceptable salt thereof is administered at a dose of about 30 mg BID.

In another embodiment, the cancer to be treated is selected from the group consisting of lung cancer, colon and rectal cancer, breast cancer, prostate cancer, liver cancer, pancreatic cancer, brain cancer, kidney cancer, ovarian cancer, stomach cancer, skin cancer, bone cancer, gastric cancer, glioma, glioblastoma, neuroblastoma, hepatocellular carcinoma, papillary renal carcinoma, head and neck squamous cell carcinoma, leukemia, lymphomas, myelomas, retinoblastoma, cervical cancer, melanoma and/or skin cancer, bladder cancer, uterine cancer, testicular cancer, esophageal cancer, and solid tumors. In some embodiments, the cancer is lung cancer, colon cancer, breast cancer, neuroblastoma, leukemia, and lymphomas. In other embodiments, the cancer is lung cancer, colon cancer, breast cancer, neuroblastoma, leukemia, or lymphoma. In a further embodiment, the cancer is non-small cell lung cancer (NSCLC) or small cell lung cancer.

In another embodiment, the cancer to be treated is a solid tumor harboring GNA11 or GNAQ mutations. In yet another embodiment, the cancer is uveal melanoma. In yet another embodiment, the cancer is uveal melanoma harboring GNA11 or GNAQ mutations. In still another embodiment, the cancer is metastatic uveal melanoma. In still another embodiment, the cancer is metastatic uveal melanoma harboring GNA11 or GNAQ mutations. In an embodiment, the cancer is cutaneous melanoma. In an embodiment, the cancer is cutaneous melanoma harboring GNA11 or GNAQ mutations.

In an embodiment, the cancer is a hematologic cancer, such as leukemia or lymphoma. In a certain embodiment, lymphoma is Hodgkin's lymphoma or Non-Hodgkin's lymphoma. In certain embodiments, leukemia is myeloid, lymphocytic, myelocytic, lymphoblastic, or megakaryotic leukemia.

In an embodiment, provided herein is Compound 1 or a pharmaceutically acceptable salt thereof and Compound 2 or a pharmaceutically acceptable salt thereof for use in therapy.

In an embodiment, provided herein is Compound 1 or a pharmaceutically acceptable salt thereof and Compound 2 or a pharmaceutically acceptable salt thereof for use in treating uveal melanoma in a patient in need thereof.

In an embodiment, provided herein is Compound 1 or a pharmaceutically acceptable salt thereof and Compound 2 or a pharmaceutically acceptable salt thereof for use in treating solid tumors harboring GNAQ or GNA11 mutations in a patient in need thereof.

In another embodiment, provided herein is Compound 1 and Compound 2 for use in treating cutaneous melanoma in a patient in need thereof.

Exemplary lengths of time associated with the course of the treatment methods disclosed herein include: about one week; two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years.

In an embodiment of the methods, the method involves the administration of a therapeutically effective amount of a combination or composition comprising compounds provided herein, or pharmaceutically acceptable salts thereof, to a subject (including, but not limited to a human or animal) in need of treatment (including a subject identified as in need).

In another embodiment of the methods, the treatment includes co-administering the amount of Compound 1 or a pharmaceutically acceptable salt thereof and the amount of Compound 2 or a pharmaceutically acceptable salt thereof. In an embodiment, the amount of Compound 1 or a pharmaceutically acceptable salt thereof and the amount of Compound 2 or a pharmaceutically acceptable salt thereof are in a single formulation or unit dosage form. In still other embodiments, the amount of Compound 1 or a pharmaceutically acceptable salt thereof and the amount of Compound 2 or a pharmaceutically acceptable salt thereof are in a separate formulations or unit dosage forms.

In the foregoing methods, the treatment can include administering the amount of Compound 1 or a pharmaceutically acceptable salt thereof and the amount of Compound 2 or a pharmaceutically acceptable salt thereof at substantially the same time or administering the amount of Compound 1 or a pharmaceutically acceptable salt thereof and the amount of Compound 2 or a pharmaceutically acceptable salt thereof at different times. In some embodiments of the foregoing methods, the amount of Compound 1 or a pharmaceutically acceptable salt thereof and/or the amount of Compound 2 or a pharmaceutically acceptable salt thereof is administered at dosages that would not be effective when one or both of Compound 1 or a pharmaceutically acceptable salt thereof and Compound 2 or a pharmaceutically acceptable salt thereof is administered alone, but which amounts are effective in combination.

In one embodiment of the foregoing methods, the treatment reduces the size of one of more lesions of the uveal melanoma or the solid tumor harboring GNAQ or GNA11 mutations in said subject.

In one embodiment of the foregoing methods, the treatment decreases the growth rate of one of more lesions of the uveal melanoma or the solid tumor harboring GNAQ or GNA11 mutations in said subject.

With respect to any of the aforementioned embodiments, Compound 1 or a pharmaceutically acceptable salt thereof is administered at a dose of about 200 mg BID and Compound 2 or a pharmaceutically acceptable salt thereof is administered at a dose of about 30 mg BID.

With respect to any of the aforementioned embodiments, Compound 1 or a pharmaceutically acceptable salt thereof is administered at a dose of about 300 mg BID and Compound 2 or a pharmaceutically acceptable salt thereof is administered at a dose of about 15 mg BID.

With respect to any of the aforementioned embodiments, Compound 1 or a pharmaceutically acceptable salt thereof is administered at a dose of about 300 mg BID and Compound 2 or a pharmaceutically acceptable salt thereof is administered at a dose of about 30 mg BID.

### Pharmaceutical Compositions

In an aspect, provided herein is a pharmaceutical composition comprising Compound 1, or a pharmaceutically acceptable salt thereof, Compound 2, or a pharmaceutically acceptable salt thereof, and at least one pharmaceutically acceptable carrier. In an embodiment, the pharmaceutical composition is for use in the treatment of uveal melanoma in a patient. In an embodiment, the pharmaceutical composition is for use in the treatment of a solid tumor harboring GNAQ or GNA11 mutations in a patient. In another embodiment, the pharmaceutical composition is for use in the treatment of cutaneous melanoma in a patient. In yet another embodiment, the uveal melanoma or cutaneous melanoma is a solid tumor harboring GNAQ or GNA11 mutations.

In an embodiment, the pharmaceutical composition reduces the size of one or more lesions of the uveal melanoma or the solid tumor harboring GNAQ or GNA11 mutations in said subject. In another embodiment, the pharmaceutical composition decreases the growth rate of one of more lesions of the uveal melanoma or the solid tumor harboring GNAQ or GNA11 mutations in said subject.

In an embodiment, Compound 1, or a pharmaceutically acceptable salt thereof, is present in the pharmaceutical composition at a dose selected from the group consisting of about 200 mg, 250 mg, 300 mg, 350 mg, 400 mg, 450 mg, 500 mg, 550 mg, and 600 mg. In another embodiment, Compound 1, or a pharmaceutically acceptable salt thereof, is present in the pharmaceutical composition at a dose of about 200 mg. In yet another embodiment, Compound 1, or a pharmaceutically acceptable salt thereof, is present in the pharmaceutical composition at a dose of about 300 mg. In still another embodiment, Compound 1, or a pharmaceutically acceptable salt thereof, is orally administered as a tablet. In another embodiment, Compound 1, or a pharmaceutically acceptable salt thereof, is administered twice daily (BID)

In an embodiment, Compound 2, or a pharmaceutically acceptable salt thereof, is present in the pharmaceutical composition at a dose selected from the group consisting of about 5 mg, 10 mg, 15 mg, 20 mg, 25 mg, 30 mg, 35 mg, 40 mg, 45 mg, 50 mg, 55 mg, and 60 mg. In another embodiment, Compound 2, or a pharmaceutically acceptable salt thereof, is present in the pharmaceutical composition at a dose of about 15 mg. In yet another embodiment, Compound 2, or a pharmaceutically acceptable salt thereof, is present in the pharmaceutical composition at a dose of about 30 mg. In still another embodiment, Compound 2, or a pharmaceutically acceptable salt thereof, is orally administered as a tablet. In another embodiment, Compound 2, or a pharmaceutically acceptable salt thereof, is administered twice daily (BID).

In an embodiment, Compound 1, or a pharmaceutically acceptable salt thereof, is present in the pharmaceutical composition at a dose of about 200 mg BID, and Compound 2, or a pharmaceutically acceptable salt thereof, is administered at a dose of about 15 mg BID. In yet another embodiment, Compound 1, or a pharmaceutically acceptable salt thereof, is present in the pharmaceutical composition at a dose of about 200 mg BID, and Compound 2, or a pharmaceutically acceptable salt thereof, is present in the pharmaceutical composition at a dose of about 30 mg BID. In another embodiment, Compound 1, or a pharmaceutically acceptable salt thereof, is present in the pharmaceutical composition at a dose of about 300 mg BID, and Compound 2, or a pharmaceutically acceptable salt thereof, is administered at a dose of about 15 mg BID. In yet another embodiment, Compound 1, or a pharmaceutically acceptable salt thereof, is present in the pharmaceutical composition at a dose of about 300 mg BID, and Compound 2, or a pharmaceutically acceptable salt thereof, is present in the pharmaceutical composition at a dose of about 30 mg BID.

In another aspect, provided herein is a pharmaceutical composition comprising Compound 1, or a pharmaceutically acceptable salt thereof, and Compound 2, or a pharmaceutically acceptable salt thereof. In another embodiment, the pharmaceutical composition of Compound 1, or a pharmaceutically acceptable salt thereof, and Compound 2, or a pharmaceutically acceptable salt thereof, further comprises one or more pharmaceutically acceptable carriers.

### Administration/Dosage/Formulations

In another aspect, provided herein is a pharmaceutical composition or pharmaceutical combination comprising the compounds disclosed herein, together with a pharmaceutically acceptable carrier.

In an embodiment of the combination product, Compound 1, or a pharmaceutically acceptable salt thereof, and Compound 2, or a pharmaceutically acceptable salt thereof, are in the same formulation. In another embodiment of the combination product, Compound 1 and Compound 2, are in separate formulations. In a further embodiment of this embodiment, the formulations are for simultaneous or sequential administration.

Administration of the combination includes administration of the combination in a single formulation or unit dosage form, administration of the individual agents of the combination concurrently but separately, or administration of the individual agents of the combination sequentially by any suitable route. The dosage of the individual agents of the combination may require more frequent administration of one of the agent(s) as compared to the other agent(s) in the combination. Therefore, to permit appropriate dosing, packaged pharmaceutical products may contain one or more dosage forms that contain the combination of agents, and one or more dosage forms that contain one of the combination of agents, but not the other agent(s) of the combination.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

The optimum ratios, individual and combined dosages, and concentrations of the drug compounds that yield efficacy without toxicity are based on the kinetics of the active ingredients' availability to target sites, and are determined using methods known to those of skill in the art.

Routes of administration of any of the compositions discussed herein include oral, nasal, rectal, intravaginal, parenteral, buccal, sublingual or topical. The compounds may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

### Kits

In an aspect, the present disclosure provides a kit for treating uveal melanoma or a solid tumor harboring GNAQ or GNA11 mutations, comprising Compound 1, or a pharmaceutically acceptable salt thereof, in a unit dosage of about 400 mg to about 600 mg, and Compound 2, or a pharmaceutically acceptable salt thereof, in a unit dosage of about 30 mg to about 60 mg. In another embodiment, the present disclosure provides a kit for treating uveal melanoma or a solid tumor harboring GNAQ or GNA11 mutations, comprising Compound 1, or a pharmaceutically acceptable salt thereof, in an amount of about 200 mg, and Compound 2, or a pharmaceutically acceptable salt thereof, in an amount of about 30 mg. In another embodiment, the present disclosure provides a kit for treating uveal melanoma or a solid tumor harboring GNAQ or GNA11 mutations, comprising Compound 1, or a pharmaceutically acceptable salt thereof, in an amount from about 400 mg per day to about 600 mg per day, and Compound 2, or a pharmaceutically acceptable salt thereof, in an amount from about 30 mg per day to about 60 mg per day. In some embodiments, the kit further comprises packaging and instructions.

In certain embodiments, the kit comprises a pharmaceutical product comprising a pharmaceutical composition comprising Compound 1, or a pharmaceutically acceptable salt thereof, and a pharmaceutically acceptable carrier or diluent; and a pharmaceutical composition comprising Compound 2, or a pharmaceutically acceptable salt thereof, and a pharmaceutically acceptable carrier or diluent.

In some embodiments, the kit comprises a pharmaceutical composition comprising Compound 1, or a pharmaceutically acceptable salt thereof; Compound 2, or a pharmaceutically acceptable salt thereof; and a pharmaceutically acceptable carrier or diluent.

In additional embodiments, pharmaceutical kits are provided. The kit includes a sealed container approved for the storage of pharmaceutical compositions, the container containing one of the above-described pharmaceutical compositions. In some embodiments, the sealed container minimizes the contact of air with the ingredients, e.g. an airless bottle. In other embodiments, the sealed container is a sealed tube. An instruction for the use of the composition and the information about the composition are to be included in the kit.

In a particular embodiment, the compounds of the combination can be dosed on the same schedule, whether by administering a single formulation or unit dosage form containing all of the compounds of the combination, or by administering separate formulations or unit dosage forms of the compounds of the combination. However, some of the compounds used in the combination may be administered more frequently than once per day, or with different frequencies that other compounds in the combination. Therefore, in one embodiment, the kit contains a formulation or unit dosage form containing all of the compounds in the combination of compounds, and an additional formulation or unit dosage form that includes one of the compounds in the combination of agents, with no additional active compound, in a container, with instructions for administering the dosage forms on a fixed schedule.

The kits provided herein include comprise prescribing information, for example, to a patient or health care provider, or as a label in a packaged pharmaceutical formulation. Prescribing information may include for example efficacy, dosage and administration, contraindication and adverse reaction information pertaining to the pharmaceutical formulation.

In all of the foregoing the combination of compounds of the invention can be administered alone, as mixtures, or with additional active agents.

A kit provided herein can be designed for conditions necessary to properly maintain the components housed therein (e.g., refrigeration or freezing). A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.).

Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings of the present disclosure as set forth.

### EXAMPLES

The compounds and methods disclosed herein are further illustrated by the following examples, which should not be construed as further limiting. The practice of the present disclosure will employ, unless otherwise indicated, conventional techniques of organic synthesis, cell biology, cell culture, and molecular biology, which are within the skill of the art.

Processes for preparing the compounds disclosed herein can be found, at least, in WO 2016/020864 and WO 2003/077914, the contents of which are incorporated in their entirety.

### Example 1: Clinical Data

Patients having metastatic uveal melanoma (MUM) were treated in a human clinical trial evaluating Compound 1 and Compound 2 at varying doses in metastatic uveal melanoma (MUM), the results of which are shown in Table 1 and Table 2.

Patient inclusion criteria:
1. Patient must be ≥18 years of age
2. Diagnosis of one of the following:
   a) MUM: Uveal melanoma with histological or cytological confirmed metastatic disease. Or
   b) Non-MUM: Advanced cutaneous melanoma, colorectal cancer, or other solid tumor that has progressed following prior standard therapies or that has no satisfactory alternative therapies and has evidence of GNAQ/11 hotspot mutation
3. Measurable disease
4. Eastern Cooperative Oncology Group≤1 and expected life expectancy of >3 months
5. Adequate organ function at screening
6. Adequate contraceptive measures for non-sterilized male and female patients of childbearing potential
7. Adequate cardiac function represented by left ventricular ejection fraction (LVEF)≥50%

Patient exclusion criteria:
1. Known symptomatic brain metastases
2. Previous treatment with a PKC inhibitor
3. Known MSI-H/dMMR tumors who have not previously received immune checkpoint inhibitors
4. Adverse events from prior anti-cancer therapy that have not resolved
5. Known acquired immunodeficiency syndrome (AIDS)-related illness, hepatitis B virus, or hepatitis C virus
6. Active infection requiring ongoing therapy
7. Recent surgery or radiotherapy
8. Prior gastrectomy or upper bowel removal or any other gastrointestinal disorder or defect
9. Females who are pregnant or breastfeeding
10. Impaired cardiac function
11. Treatment with prohibited medications that cannot be discontinued prior to study entry
12. For patients receiving IDE 196 powder-in-capsule (PIC) formulation, allergy to mammalian meat products and gelatin
13. Prior treatment with a MEK inhibitor
14. History or current evidence of retinal vein occlusion (RVO) or current risk factors for RVO
15. History of interstitial lung disease
16. History of thromboembolic or cerebrovascular events ≤12 weeks prior to first dose
17. Concurrent neuromuscular disorders that are associated with elevated creatine phosphokinase (CPK)
18. Uncontrolled arterial hypertension despite medical treatment
19. Allergy to binimetinib or its components
20. History of syncope

TABLE 1

| | | | Tumor size from | |
|---|---|---|---|---|
| | Dose: | | baseline | |
| | Compound | | (determined from | |
| | 1 (mg)/ | | post baseline | |
| Patient | Compound | Mutation | scan) (Best of % | |
| ID | 2 (mg) | status | Change RECIST) | Dosing status |
| 1** | 300/15 | GNA11 | +60% | >1 Dose |
| | | (R183Y) | | Interruption |
| 2* | 200/30 | # | +24.3% | No Dose |
| | | | | Interruption |
| 3** | 300/15 | GNA11 | +13.6% | >1 Dose |
| | | (Q209L) | | Interruption |
| 4** | 300/30 | GNAQ | −5.5% | No Dose |
| | | (Q290L) | | Interruption |
| 5* | 200/30 | # | −7.7% | No Dose |
| | | | | interruption |
| 6* | 200/30 | # | −14.7% | No Dose |
| | | | | Interruption |
| 7** | 300/15 | GNA11 | −18.3% | >1 Dose |
| | | (Q209L) | | Interruption |
| 8* | 200/30 | # | −18.9% | No Dose |
| | | | | interruption |
| 9* | 200/30 | # | −20.5% | No Dose |
| | | | | interruption |
| 10** | 300/15 | GNA11 | −22.4% | >1 Dose |
| | | (R183C) | | Interruption |
| 11** | 300/30 | # | −24.4% | >1 Dose |
| | | | | Interruption |
| 12** | 300/15 | GNA11 | −26.2% | >1 Dose |
| | | (Q290L) | | Interruption |
| 13** | 300/15 | # | −31.0% | >1 Dose |
| | | | | Interruption |
| 14** | 300/30 | # | −40.5% | >1 Dose |
| | | | | Interruption |

**2 or more post baseline scans
*1 post baseline scan
# determination of mutational status pending; wherein “Dose Interruption” is defined as at least one missed dose of either Compound 1 and/or Compound 2 over at least one 7-day dosing cycle.

### Preliminary Data Summary (Table 1)

i. Two partial responses were observed out of nine metastatic uveal melanoma (MUM) patients with at least 2 post-baseline scans (22%) by RECIST 1.1 guidelines. These included 1 confirmed partial response (−31.1%) and 1 unconfirmed partial response (−40.5%) awaiting a confirmatory scan. The unconfirmed partial response was confirmed as partial response as of Mar. 25, 2022.
ii. 79% (n=11) of evaluable MUM patients (n=14) with at least 1 post-baseline scan showed tumor reduction
iii. Combination doses for Phase 1/2 dose expansion cohort of the clinical trial were selected to be 200 mg BID of Compound 1 in combination with 30 mg BID of Compound 2 based on evidence of activity and overall tolerability in a larger treatment cohort, including tolerability relative to other evaluated dosing regimens (e.g., as evidenced by ability to continue dosing without dose interruption over a treatment period.
iv. The observed treatment-related adverse events observed in the combination therapy comprising administration of Compound 1 and Compound 2 included nausea, vomiting, diarrhea, rash, edema, AST/ALT increase and CK increase (>10%), and transient hypotension (<10%).

### Benefit of the Combination Dosing Regimen

a. Combination dosing and schedule of Compound 1 and Compound 2 were adjusted to modulate common GI side effects of nausea, vomiting, and diarrhea, as well as minimization of acneiform rash and transient hypotensive episodes; a majority of patients receiving the expansion cohort dosing regimen of 200 mg BID Compound 1 and 30 mg BID Compound 2 were able to continue administration of the combination therapy without dose interruption, including over multiple uninterrupted dosing cycles comprising at least 7 days, as reflected in Table 1.

### Data Analysis—Importance of Uninterrupted Dosing

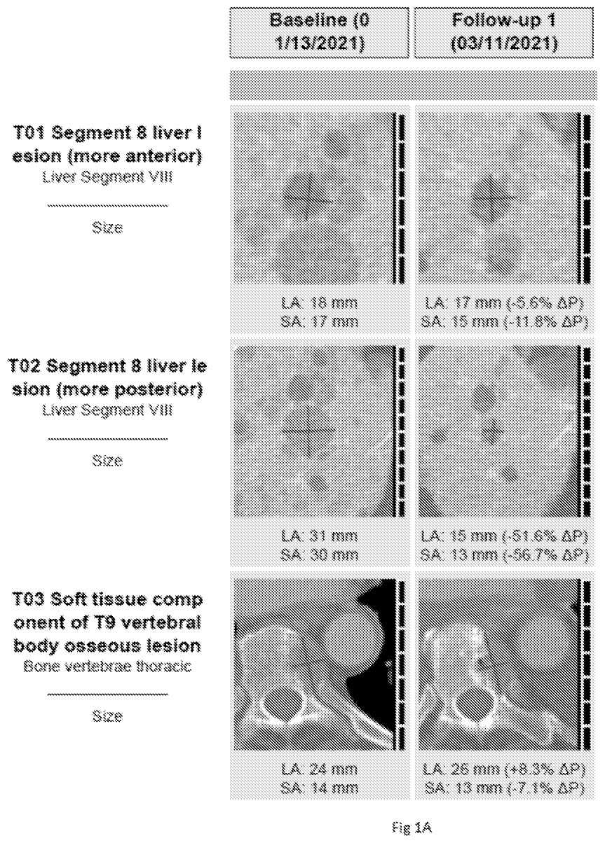

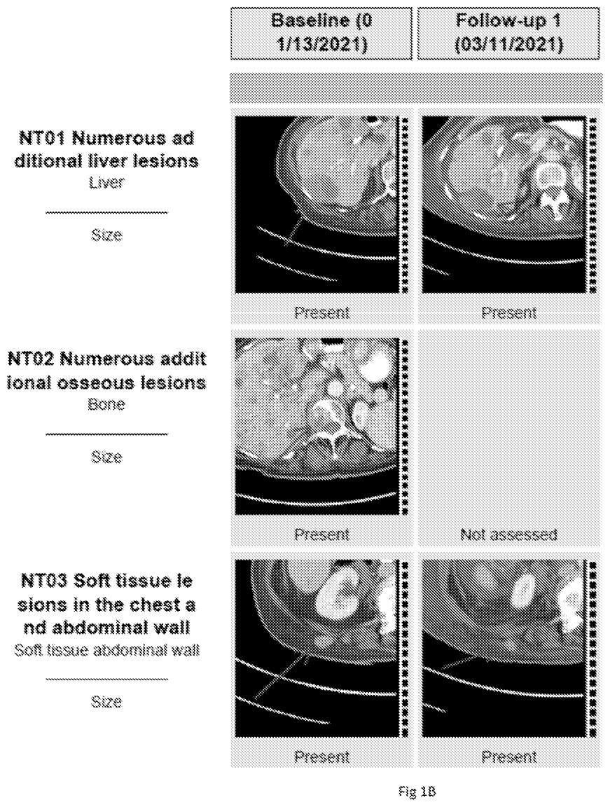

a. Table 1 shows that 75% of a subset of patients (n=3 out of n=4) who were administered with 200 mg of Compound 1 and 30 mg of Compound 2 and who experienced a decrease in tumor size as measured in one or more clinically selected lesions relative to post-baseline scan, had no dose interruption for at least 28 days.
b. FIG. 1A and FIG. 1B show CT scans for patient 9, who, upon treatment with Compound 1 and Compound 2, experienced a 20.5% reduction in some of target lesions.

TABLE 2

| | | | RECIST |
|---|---|---|---|
| | Dose: Compound 1 | | Best % |
| Patient | (mg) BID/Compound | Mutation | Change from |
| ID | 2 (mg) BID | status | Baseline |
| 3 | 300/15 | GNA11(Q209L) | 13.64% |
| 10 | 300/15 | GNA11(R183C) | −22.45% |
| 7 | 300/15 | GNA11(Q209L) | −18.29% |
| 12 | 300/15 | GNA11(Q209L) | −29.13% |
| 1 | 300/15 | GNA11(R183Y) | 60.00% |
| 13 | 300/15 | unknown | −30.99% |
| 11 | 300/30 | GNA11(Q209L) | −24.44% |
| 4 | 300/30 | GNAQ(Q209L) | −16.36% |
| 14 | 300/30 | GNAQ (Q209P) | −68.10% |
| 9 | 200/30 | GNAQ (Q209P) | −20.55% |
| 5 | 200/30 | GNAQ (Q209L) | −7.69% |
| 8 | 200/30 | GNA11(Q209L) | −18.87% |
| 2 | 200/30 | GNAQ (Q209P) | 24.29% |
| 15 | 200/30 | # | 60.81% |
| 6 | 200/30 | # | −14.71% |
| 16* | 300/30 | GNA11(Q209L) | 2.08% |
| 17* | 300/30 | GNA11(R183Y) | −12.30% |
| 18* | 300/30 | GNA11(Q209L) | 10.96% |
| 19* | 300/30 | GNAQ(Q209L) | −8.87% |
| 20* | 200/30 | GNA11(Q209L) | −15.79% |
| 21 | 200/30 | GNAQ(Q209L) | −4.12% |
| 22* | 200/30 | GNA11(Q209L) | −12.50% |
| 23** | 200/30 | # | −47.75% |
| 24** | 200/30 | # | −21.54% |
| 25** | 300/30 | # | 15.00% |
| 26** | 300/30 | GNA11(Q209L) | 28.08% |
| 27** | 300/30 | GNAQ (Q209P) | −46.00% |
| 28** | 300/30 | # | −7.69% |
| 29** | 300/30 | GNA11 Q209L) | −29.49% |

*Patients are administered Compound 1 and Compound 2 according to intermittent dosing schedule
**Patients are administered Compound 1 and Compound 2 according to run-in dosing schedule
All other patients are administered with Compound 1 and Compound 2 every day in at least one 7-day dosing cycle
# Determination of mutational status pending

As of Mar. 25, 2022, Compound 1 (300 mg) and Compound 2 (15 mg) combination (n=6 patients) showed a disease control rate of 66.67% (n=6 patients) as measured by best target lesion response, wherein disease control rate (DCR) included complete response (CR), partial response (PR), and stable disease (SD). Overall response rate was 16.67% (n=1 patient).

As of Mar. 25, 2022, Compound 1 (200 mg) and Compound 2 (30 mg) combination (n=16 patients) showed a disease control rate of 72.73% (n=11 patients) as measured by best target lesion response, wherein disease control rate (DCR) included complete response (CR), partial response (PR), and stable disease (SD). Overall response rate was 9.09% (n=1 patient).

As of Mar. 25, 2022, Compound 1 (300 mg) and Compound 2 (30 mg) combination (n=14 patients) showed a disease control rate of 83.33% (n=12 patients) as measured by best target lesion response, wherein disease control rate (DCR) included complete response (CR), partial response (PR), and stable disease (SD). Overall response rate was 8.33% (n=1 patient).

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Where a conflict exists between the instant application and a reference provided herein, the instant application shall dominate. Other embodiments are within the following claims.

1. A method of treating uveal melanoma or a solid tumor harboring GNAQ or GNA11 mutations in a subject in need thereof, comprising administering to the subject Compound 1: 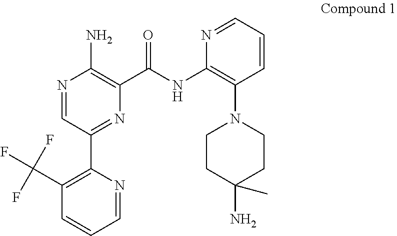 [SMILES: CC1(N)CCN(c2cccnc2NC(=O)c2nc(-c3ncccc3C(F)(F)F)cnc2N)CC1]
or a pharmaceutically acceptable salt thereof,
and Compound 2: 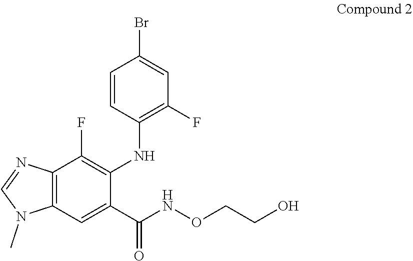 [SMILES: Cn1cnc2c(F)c(Nc3ccc(Br)cc3F)c(C(=O)NOCCO)cc21]
or a pharmaceutically acceptable salt thereof;
wherein
Compound 1 or a pharmaceutically acceptable salt thereof is administered at a dose of about 200 mg two times per day (BID); and
Compound 2 or a pharmaceutically acceptable salt thereof is administered at a dose of about 30 mg BID.

2. A method of treating uveal melanoma or a solid tumor harboring GNAQ or GNA11 mutations in a subject in need thereof, comprising administering to the subject Compound 1: 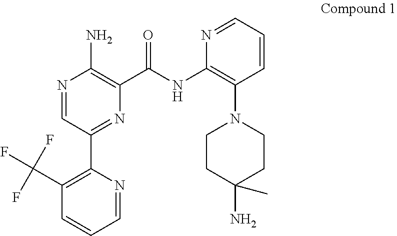 [SMILES: CC1(N)CCN(c2cccnc2NC(=O)c2nc(-c3ncccc3C(F)(F)F)cnc2N)CC1]
or a pharmaceutically acceptable salt thereof,
and Compound 2: 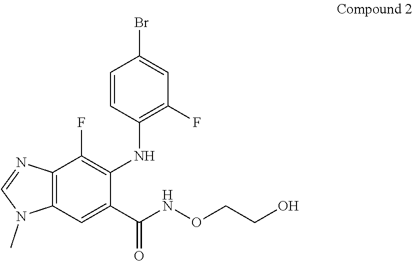 [SMILES: Cn1cnc2c(F)c(Nc3ccc(Br)cc3F)c(C(=O)NOCCO)cc21]
or a pharmaceutically acceptable salt thereof;
wherein
Compound 1, or a pharmaceutically acceptable salt thereof, is administered at a dose of 400 mg to 600 mg daily; and
Compound 2, or a pharmaceutically acceptable salt thereof, is administered at a dose of 30 mg to 60 mg daily.

3. A method of treating uveal melanoma or a solid tumor harboring GNAQ or GNA11 mutations in a subject in need thereof, comprising administering to the subject Compound 1: 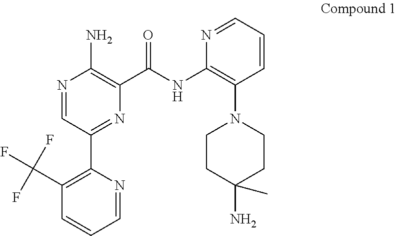 [SMILES: CC1(N)CCN(c2cccnc2NC(=O)c2nc(-c3ncccc3C(F)(F)F)cnc2N)CC1]
or a pharmaceutically acceptable salt thereof,
and Compound 2: 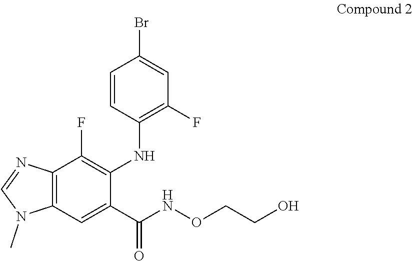 [SMILES: Cn1cnc2c(F)c(Nc3ccc(Br)cc3F)c(C(=O)NOCCO)cc21]
or a pharmaceutically acceptable salt thereof;
according to a dosing schedule comprising at least one 7-day dosing cycle, wherein
Compound 1 or a pharmaceutically acceptable salt thereof is administered at a dose of about 200 mg BID every day of the at least one 7-day dosing cycle; and
Compound 2 or a pharmaceutically acceptable salt thereof is administered at a dose of about 30 mg BID for five consecutive days and then not administered for two consecutive days of the at least one 7-day dosing cycle.

4. A method of treating uveal melanoma or a solid tumor harboring GNAQ or GNA11 mutations in a subject in need thereof, comprising administering to the subject Compound 1 or a pharmaceutically acceptable salt thereof, and Compound 2 or a pharmaceutically acceptable salt thereof;
wherein
Compound 1, or a pharmaceutically acceptable salt thereof, is administered at a dose of about 400 mg to about 600 mg daily; and
Compound 2, or a pharmaceutically acceptable salt thereof, is administered at a dose of about 30 mg to about 60 mg daily.

5. A method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;
according to a dosing schedule comprising at least one 7-day dosing cycle,
wherein Compound 1 or a pharmaceutically acceptable salt thereof is administered at a dose of about 200 mg BID every day to about 300 BID every day of the at least one 7-day dosing cycle; and Compound 2 or a pharmaceutically acceptable salt thereof is administered at a dose of about 15 mg BID every day to about 30 mg BID every day of the at least one 7-day dosing cycle.

6. A method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;
according to a dosing schedule comprising at least two 7-day dosing cycles,
wherein Compound 1 or a pharmaceutically acceptable salt thereof is administered at a dose of about 200 mg BID every day to about 300 BID every day of the at least two 7-day dosing cycles; and Compound 2 or a pharmaceutically acceptable salt thereof is administered at a dose of about 15 mg BID every day to about 30 mg BID every day starting on the 8^{th} day of the at least two 7-day dosing cycles.

7. A method of treating uveal melanoma or a solid tumor harboring GNAQ or GNA11 mutations in a subject in need thereof, comprising administering to the subject Compound 1, or a pharmaceutically acceptable salt thereof, and Compound 2, or a pharmaceutically acceptable salt thereof;
according to a dosing schedule comprising at least one 7-day dosing cycle,
wherein Compound 1 or a pharmaceutically acceptable salt thereof is administered at a dose of about 200 mg BID every day to about 300 BID every day of the at least one 7-day dosing cycle; and Compound 2 or a pharmaceutically acceptable salt thereof is administered at a dose of about 15 mg BID every day to about 30 mg BID for five consecutive days and then not administered for two consecutive days of the at least one 7-day dosing cycle.

8. The method of any one of claims 4 to 7, wherein Compound 1, or a pharmaceutically acceptable salt thereof, is administered at a dose of about 200 mg two times per day (BID); and Compound 2, or a pharmaceutically acceptable salt thereof, is administered at a dose of about 30 mg two times per day (BID).

9. The method of any one of claims 4 to 7, wherein Compound 1, or a pharmaceutically acceptable salt thereof, is administered at a dose of about 300 mg two times per day (BID); and Compound 2, or a pharmaceutically acceptable salt thereof, is administered at a dose of about 30 mg two times per day (BID).

10. The method of any one of claims 4 to 7, wherein Compound 1, or a pharmaceutically acceptable salt thereof, is administered at a dose of about 300 mg two times per day (BID); and Compound 2, or a pharmaceutically acceptable salt thereof, is administered at a dose of about 15 mg two times per day (BID).

11. The method of any one of claims 1-10, wherein the uveal melanoma is metastatic uveal melanoma.

12. The method of any one of claims 1-11, wherein the uveal melanoma is a solid tumor harboring GNAQ or GNA11 mutations.

13. The method of any one of claims 1-10, wherein the solid tumor harboring GNAQ or GNA11 mutations is cutaneous melanoma.

14. The method of any one of claims 1-8 and 11-13, wherein Compound 1, or a pharmaceutically acceptable salt thereof, is administered at a dose of 200 mg BID.

15. The method of any one of claims 1-8 and 11-13, wherein Compound 2, or a pharmaceutically acceptable salt thereof, is administered at a dose of 30 mg BID.

16. The method of any one of claims 1-4, 7-9, and 11-15, wherein according to a dosing schedule comprising at least one 7-day dosing cycle, Compound 2 or a pharmaceutically acceptable salt thereof is administered at a dose of about 30 mg BID for five consecutive days and then not administered for two consecutive days of the at least one 7-day dosing cycle.

17. The method of any one of claims 1-16, wherein Compound 1, or a pharmaceutically acceptable salt thereof, and Compound 2, or a pharmaceutically acceptable salt thereof, are administered in a single formulation.

18. The method of claim 17, wherein Compound 1, or a pharmaceutically acceptable salt thereof, and Compound 2, or a pharmaceutically acceptable salt thereof, are administered in a single formulation further comprising one or more pharmaceutically acceptable carriers.

19. The method of any one of claims 1-16, wherein Compound 1, or a pharmaceutically acceptable salt thereof, and Compound 2, or a pharmaceutically acceptable salt thereof, are administered separately.

20. The method of any one of claims 1-16 and 19, wherein the treatment comprises administering the Compound 1, or a pharmaceutically acceptable salt thereof, and Compound 2, or a pharmaceutically acceptable salt thereof, at substantially the same time.

21. The method of any one of claims 1-16 and 19, wherein the treatment comprises administering Compound 1, or a pharmaceutically acceptable salt thereof, and Compound 2, or a pharmaceutically acceptable salt thereof, at different times.

22. The method of claim 21, wherein Compound 1, or a pharmaceutically acceptable salt thereof, is administered to the subject, followed by administration of Compound 2, or a pharmaceutically acceptable salt thereof.

23. The method of claim 21, wherein Compound 2, or a pharmaceutically acceptable salt thereof, is administered to the subject, followed by administration of Compound 1, or a pharmaceutically acceptable salt thereof.

24. The method of any one of claims 1-23, wherein administering Compound 1, or a pharmaceutically acceptable salt thereof, and Compound 2, or a pharmaceutically acceptable salt thereof, reduces the size of one or more lesions of the uveal melanoma or the solid tumor harboring GNAQ or GNA11 mutations in said subject.

25. The method of any one of claims 1-23, wherein administering Compound 1, or a pharmaceutically acceptable salt thereof, and Compound 2, or a pharmaceutically acceptable salt thereof, decreases the growth rate of one of more lesions of the uveal melanoma or the solid tumor harboring GNAQ or GNA11 mutations in said subject.

26. The method of any one of claims 1-25, comprising administering to the subject in need thereof Compound 1:

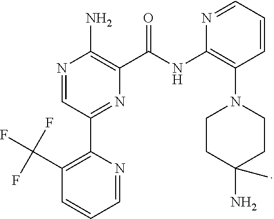 [SMILES: CC1(N)CCN(c2cccnc2NC(=O)c2nc(-c3ncccc3C(F)(F)F)cnc2N)CC1]

27. The method of any one of claims 1-26, comprising administering to the subject in need thereof Compound 2:

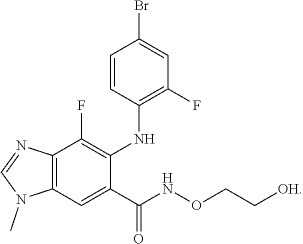 [SMILES: Cn1cnc2c(F)c(Nc3ccc(Br)cc3F)c(C(=O)NOCCO)cc21]

28. The method of any one of claims 1-27, wherein Compound 1, or a pharmaceutically acceptable salt thereof, and Compound 2, or a pharmaceutically acceptable salt thereof, are administered orally.

29. A combination product comprising
(i) about 200 mg Compound 1: 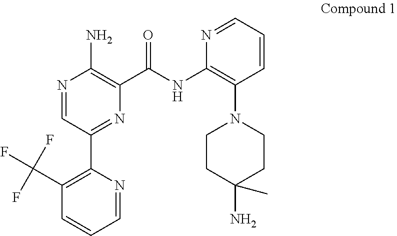 [SMILES: CC1(N)CCN(c2cccnc2NC(=O)c2nc(-c3ncccc3C(F)(F)F)cnc2N)CC1]
or a pharmaceutically acceptable salt thereof; and
(ii) about 30 mg of Compound 2: 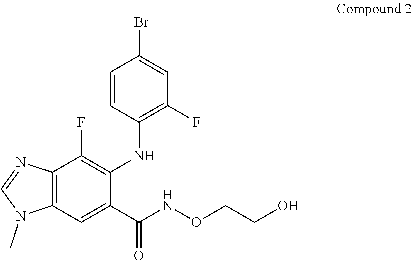 [SMILES: Cn1cnc2c(F)c(Nc3ccc(Br)cc3F)c(C(=O)NOCCO)cc21]
or a pharmaceutically acceptable salt thereof.

30. A combination product comprising
(i) 200 mg to 600 mg Compound 1: 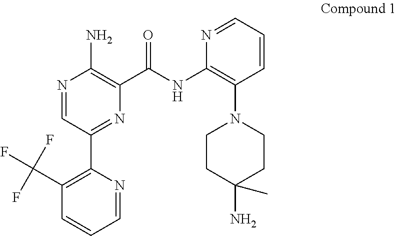 [SMILES: CC1(N)CCN(c2cccnc2NC(=O)c2nc(-c3ncccc3C(F)(F)F)cnc2N)CC1]
or a pharmaceutically acceptable salt thereof; and
(ii) 15 mg to 60 mg of Compound 2: 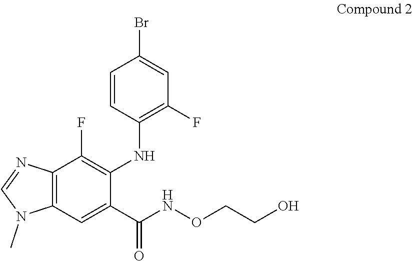 [SMILES: Cn1cnc2c(F)c(Nc3ccc(Br)cc3F)c(C(=O)NOCCO)cc21]
or a pharmaceutically acceptable salt thereof.

31. The combination product of claim 30, wherein the combination product comprises 200 mg of Compound 1, or a pharmaceutically acceptable salt thereof; and 30 mg of Compound 2, or a pharmaceutically acceptable salt thereof.

32. The combination product of claim 30, wherein the combination product comprises 300 mg of Compound 1, or a pharmaceutically acceptable salt thereof; and 30 mg of Compound 2, or a pharmaceutically acceptable salt thereof.

33. The combination product of claim 30, wherein the combination product comprises 300 mg of Compound 1, or a pharmaceutically acceptable salt thereof; and 15 mg of Compound 2, or a pharmaceutically acceptable salt thereof.

34. The combination product of claim 29, wherein the combination product comprises about 200 mg of Compound 1, or a pharmaceutically acceptable salt thereof.

35. The combination product of claim 29 or 34, wherein the combination product comprises about 30 mg of Compound 2, or a pharmaceutically acceptable salt thereof.

36. The combination product of any one of claims 29-35, comprising Compound 1:

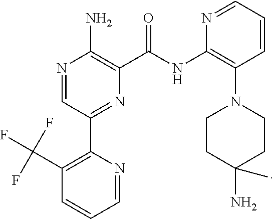 [SMILES: CC1(N)CCN(c2cccnc2NC(=O)c2nc(-c3ncccc3C(F)(F)F)cnc2N)CC1]

37. The combination product of any one of claims 29-36, comprising Compound 2: 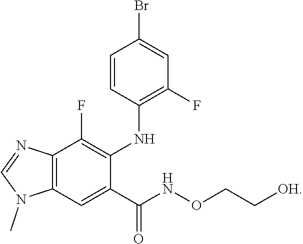 [SMILES: Cn1cnc2c(F)c(Nc3ccc(Br)cc3F)c(C(=O)NOCCO)cc21]
